(19) United States
(12) Patent Application Publication
(10) Pub. No.: US 2022/0241030 A1
(43) Pub. Date: Aug. 4, 2022

(54) WEARABLE SURGICAL ROBOT ARM

(71) Applicant: Sam Youl YOON, Seoul (KR)

(72) Inventor: So Jin YOON, Suwon-si, Gyeonggi-do (KR)

(73) Assignee: Sam Youl YOON, Seoul (KR)

(21) Appl. No.: 17/288,580

(22) PCT Filed: Jul. 31, 2019
(86) PCT No.: PCT/KR2019/009569

§ 371 Date: Nov. 9, 2021

(30) Foreign Application Priority Data
Oct. 26, 2018 (KR) 10-2018-0129218

(51) Int. Cl. A61B 34/30; A61B 34/00

(52) U.S. Cl. CPC A61B 34/30; G10L 17/22; A61B 2560/0493; A61B 34/25

(57) ABSTRACT

The present disclosure relates to a wearable surgical robot arm comprising: a base configured to move together with an arm of an operator; a motion part that has a plurality of accommodating grooves in which a surgical instrument is be mounted and that is moveably installed on the base such that one of the plurality of accommodating grooves is positioned in a first position; a shaft that is disposed such that the shaft moves forward or backward to selectively penetrate the accommodating groove positioned in the first position at a rear of the motion part; a first driver that moves the motion part such that one surgical instrument selected from the surgical instruments mounted in the plurality of accommodating grooves moves to the first position; a second driver that moves the shaft to move the surgical instrument positioned in the first position to a second position positioned in front of the first position; and a third driver that provides driving force to the surgical instrument that moved to the second position.

### 1. FIELD

The present disclosure relates to a wearable surgical robot arm, and more specifically, to a wearable surgical robot arm capable of proceeding a surgery through a surgical instrument that may be controlled by manipulation of an operator while the robot arm is fixed to an arm of the operator.

### 2. BACKGROUND

Medically, a surgery refers to curing a disease by cutting, incising, or performing manipulation on skin, mucous membranes, and other tissues using a surgical instrument. Especially, an open surgery and laparoscopic surgery, in which the skin of a surgical site is cut open and then the internal organs therein are treated, formed, or removed, have problems of bleeding, adverse effects, patient's pain, scar, and precision of the surgery, and thus, in recent days, surgeries using robots are getting the spotlight as an alternative.

Conventional surgical robots, like the Da Vinci Surgical System developed by Intuitive Surgical, USA, consist of a master robot that generates and transmits a necessary signal by manipulation of a doctor and a slave robot that receives the signals from the master robot and directly applies a necessary manipulation for the surgery. Generally, the slave robot is installed in a surgery room while the master robot is installed in a manipulating room, that is separate from the surgery room, respectively, and the master robot and the slave robot are connected by wired or wireless methods to proceed remote operation.

However, conventional surgical robots have very large volumes and require considerable amount of space for installation, and the configuration of the device is complicated, and thus it takes a lot of time and cost in manufacturing, installing and learning the process. Especially, even in the case of a simple surgery, when using a conventional surgical robot, there is a problem that the robot surgery might become an inefficient method due to the time and cost being spent.

Further, when there is a need to use other surgical instruments during a procedure, a method is used where the position of the robot arm of the surgical robot currently being used is moved, and the surgical instrument is replaced by a surgical assistant and then moved back to the original position. In this case, however, the surgical assistant has to move the robot arm to a space in which the surgical instrument can be replaced, and thus it may cause unnecessary movement line of the robot arm. This is a problem in that unnecessary time is wasted considering that the nature of surgeries is to complete the procedure as quickly as possible. Further, there is a problem that additional workforce (1-2 surgical assistant nurses, 1 instrument delivery nurse) is necessary for a simple work of replacing the surgical instruments.

Further, in a case where an unexpected emergency occurs to the patient during the surgery procedure using a robot, the doctor who was performing the surgery using the master robot in a remote location separated from the surgery room needs preparation time to directly participate in the surgery, thus the surgery time is inevitably delayed, which makes it impossible to immediately respond to the emergency situation.

### SUMMARY

Therefore, a purpose of the present disclosure is to resolve the problems of prior art, that is, to provide a wearable surgical robot arm that enables immediate response by the operator even when an unexpected emergency situation occurs, since a surgery is proceeded through a surgical instrument that is controlled by manipulation of the operator with the robot arm fixed to the arm of the operator.

Further, another purpose of the present disclosure is to provide a wearable surgical robot arm that may not only reduce the surgery time but also does not require additional workforce for replacing the surgical instrument, as the base mounted onto the operator's arm has a motion part onto which a plurality of surgical instruments are mounted, and replacement of the surgical instrument is made according to signals being input by the operator.

Further, another purpose of the present disclosure is to provide a wearable surgical robot arm that may prevent fatigue of the operator's arm from being increased by the self-weight of the robot arm, since the base mounted onto the operator's arm is supported against a structure by a multi-joint support arm.

The aforementioned purpose may be achieved according to the present disclosure by a wearable surgical robot arm comprising: a base configured to move together with an arm of an operator; a motion part that has a plurality of accommodating grooves in which a surgical instrument is be mounted and that is moveably installed on the base such that one of the plurality of accommodating grooves is positioned in a first position; a shaft that is disposed such that the shaft moves forward or backward to selectively penetrate the accommodating groove positioned in the first position at a rear of the motion part; a first driver that moves the motion part such that one surgical instrument selected from the surgical instruments mounted in the plurality of accommodating grooves moves to the first position; a second driver that moves the shaft to move the surgical instrument positioned in the first position to a second position positioned in front of the first position; and a third driver that provides driving force to the surgical instrument that moved to the second position.

Here, it is desirable that the motion part is rotatably disposed on the base.

Further, it is desirable that the third driver is provided in the shaft.

Further, it is desirable that at a front end of the shaft, a driving wheel is provided for transmitting the driving force being provided from the third driver, and at a rear end of the surgical instrument, a following wheel is provided in a position correspond to the driving wheel to interlock with the driving wheel.

Further, it is desirable that the base is provided with an inputter by which the operator may input a control signal.

Further, it is desirable that the inputter includes a first inputter for inputting a control signal for controlling operations of the surgical instrument.

Further, it is desirable that the inputter includes a second inputter for inputting a control signal for driving the first driver and the second driver.

Further, it is desirable that the second inputter includes a voice inputter for recognizing voice of the operator, and a voice recognizer for generating a control signal for driving the first driver and the second driver according to the voice input through the voice inputter.

Further, it is desirable that the wearable surgical robot arm further includes a multi-joint support arm that supports the base in a moveable form.

According to the present disclosure, a wearable surgical robot arm is provided, that enables immediate response by the operator even when an unexpected emergency situation occurs, since a surgery is proceeded through a surgical instrument that is controlled by manipulation of the operator with the robot arm fixed to the arm of the operator.

Further, a wearable surgical robot arm is provided, that may not only reduce the surgery time but also does not require additional workforce for replacing the surgical instrument, as the base mounted onto the operator's arm has a motion part onto which a plurality of surgical instruments are mounted, and replacement of the surgical instrument is made according to signals being input by the operator.

Further, a wearable surgical robot arm is provided, that may prevent fatigue of the operator's arm being increased by the self-weight of the robot arm, since the base mounted onto the operator's arm is supported against a structure by a multi-joint support arm.

### BRIEF DESCRIPTION OF THE DRAWINGS

FIG. 1 is a schematic configuration view of a wearable surgical robot art of the present disclosure;

FIG. 2 is a perspective view of the wearable surgical robot arm of the present disclosure;

FIG. 3 is a function view illustrating operations of a motion part according to the wearable surgical robot arm of the present disclosure;

FIG. 4 is a function view illustrating operations of a shaft according to the wearable surgical robot arm of the present disclosure;

FIG. 5 is a perspective view illustrating a coupling site of the shaft and a surgical instrument according to the wearable surgical robot arm of the present disclosure; and

FIG. 6 is a view illustrating a modified example of the motion part according to the wearable surgical robot arm of the present disclosure.

### DETAILED DESCRIPTION

Prior to the detailed description, in numerous embodiments, it is to be noted that regarding components having the same configuration, explanation will be made with reference to a first embodiment as a representative using the same reference numerals, and in the rest of the embodiments, explanation will be made regarding configurations that are different from the first embodiment.

Hereinbelow, with reference to the drawings attached, detailed explanation will be made on a wearable surgical robot arm according to a first embodiment of the present disclosure.

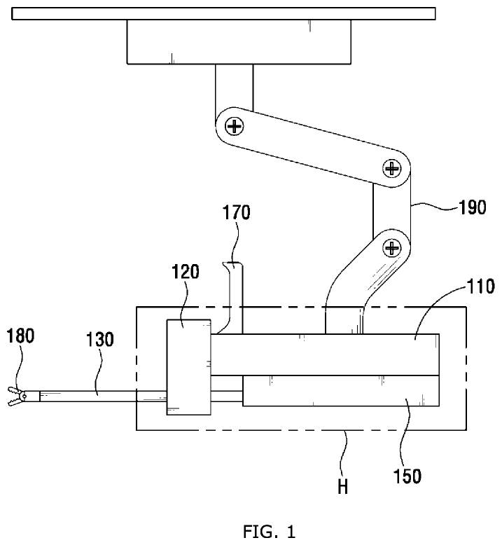

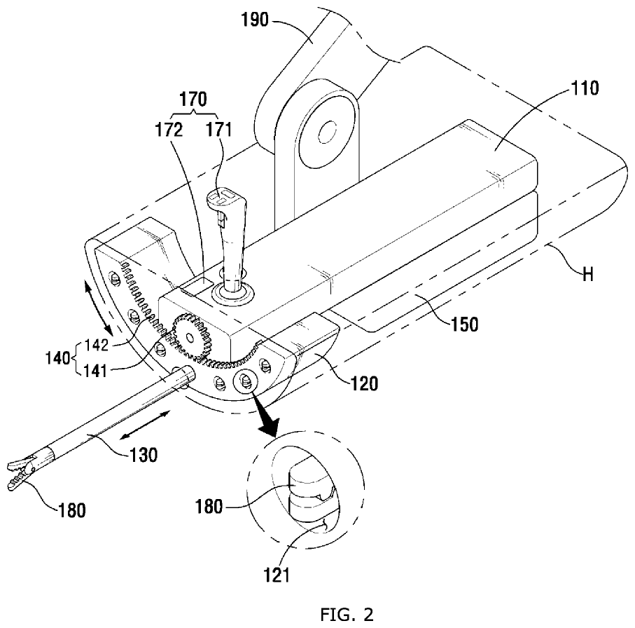

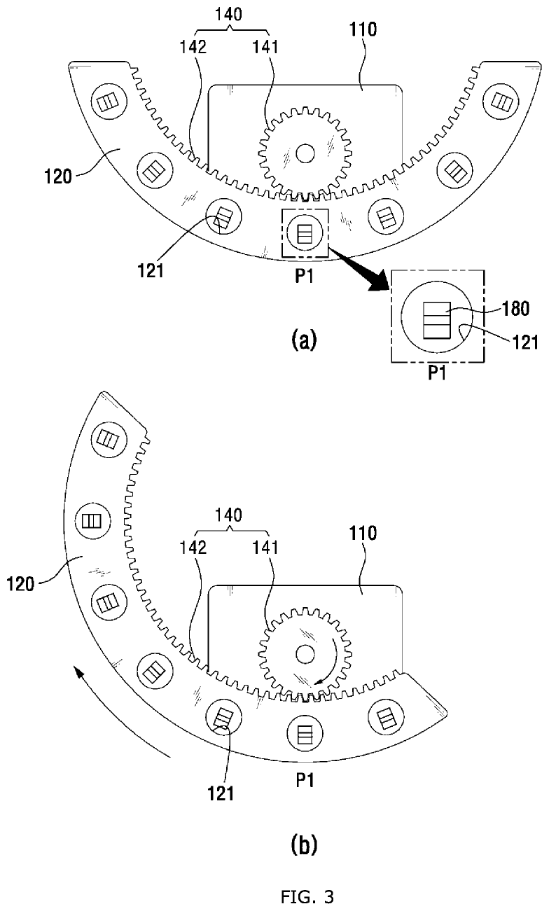

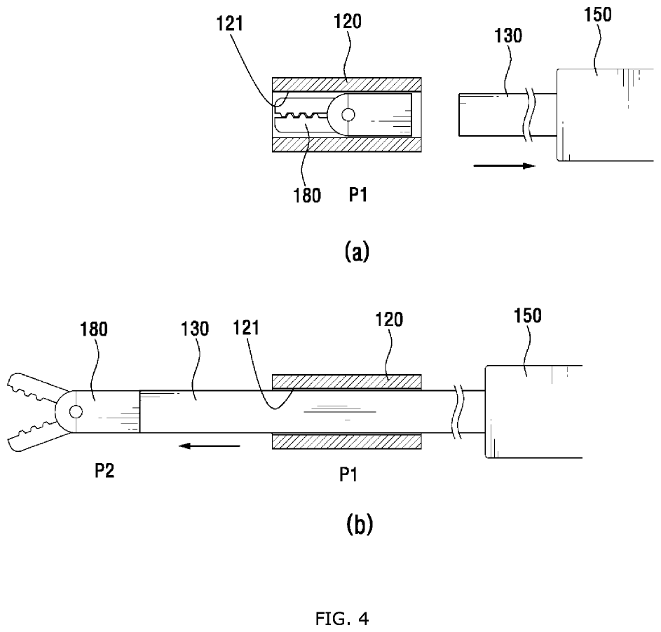

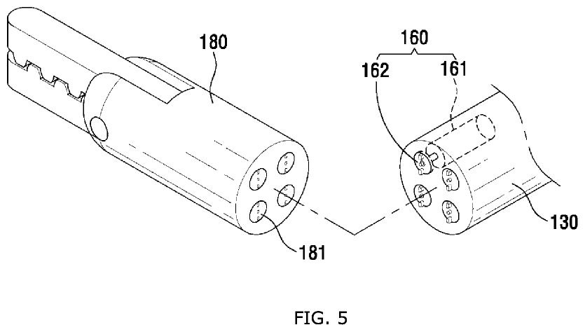

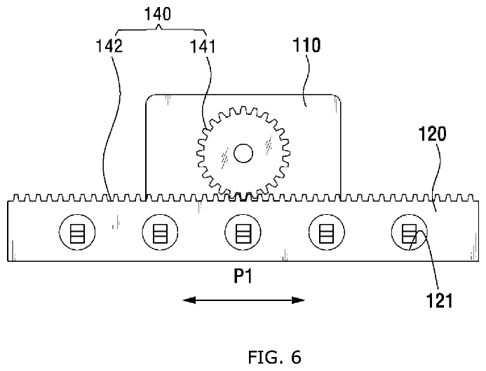

Of the attached drawings, FIG. 1 is a schematic configuration view of a wearable surgical robot art of the present disclosure, FIG. 2 is a perspective view of the wearable surgical robot arm of the present disclosure, FIG. 3 is a function view illustrating operations of a motion part according to the wearable surgical robot arm of the present disclosure, FIG. 4 is a function view illustrating operations of a shaft according to the wearable surgical robot arm of the present disclosure, FIG. 5 is a perspective view illustrating a coupling site of the shaft and a surgical instrument according to the wearable surgical robot arm of the present disclosure, and FIG. 6 is a view illustrating a modified example of the motion part according to the wearable surgical robot arm of the present disclosure.

A wearable surgical robot arm as that illustrated in the aforementioned drawings includes a base 110, a motion part 120, a shaft 130, a first driver 140, a second driver 150, a third driver 160, an inputter 170, a surgical instrument 180, and a multi-joint support arm 190.

Meanwhile, in order to prevent a complicated mechanical configuration from being exposed outside from a surgery room, it will be preferable to have a housing H for accommodating the base 110, the motion part 120, the first driver 140, and the second driver 150.

The base 110 is configured to be mounted onto an arm of an operator such as a surgeon, to move together with the operator's arm, and may have a band or a belt to be fixed to the arm, or it may be configured in a form to which the arm may be fitted.

The motion part 120 may be rotatably installed in the base 110 and may consist of arc-shaped cross sections. The motion part have a plurality of accommodating grooves 121 on the same radius about the center of rotation and a surgical instrument 180 may be mounted in the accommodating groove. The motion part may enable one of the plurality of accommodating grooves 121 to be positioned in a first position P1, according to the rotation position. Meanwhile, although not illustrated in the drawings, the motion part 120 may have an arc-shaped rail along the motion direction of the motion part 120, and the base 110 may have a block to be coupled to the rail so that rotation of the motion part 120 may be guided. Meanwhile, although in the present embodiment an example was explained that as the motion part 120 rotates, one of the plurality of accommodating grooves 121 moves to the first position P1, it will also be possible to configure such that as the motion part 120 moves left/right (or up/down), one of the plurality of accommodating grooves 121 moves to the first position P1 as illustrated in FIG. 6.

The shaft 130 is disposed on a same axis line as the accommodating groove 121 of the first position P1 at a rear of the motion part 120, and the shaft is supported against the base 110 so as to axially move back and forth. Meanwhile, although not illustrated in the drawings, at a front end of the shaft 130, a coupling instrument that may selectively fix or unfix a rear end of the surgical instrument 180 just as a gripper, may be provided.

The first driver 140 is for rotating the motion part 120 so that any one surgical instrument 180 selected from among the surgical instruments 180 mounted onto the plurality of accommodating grooves 121 can move to the first position P1, and the first driver may include a servo motor (not illustrated) provided in the base 110, a pinion 141 fixed to a driving axis of the servo motor, and an arc-shaped rake 142 that may be provided in the motion part 120 to interlock with the pinion 141. Meanwhile, in the present embodiment, explanation was made based on an example of rotating the motion part 120 using the pinion 141 and the rake 142, but there is no limitation thereto, and thus, various forms of driving means and power transmission mechanism that can control the rotation position of the moving part 120 may be applied.

The second driver 150 is for moving the shaft 130 forward and backward in an axial direction, and may be configured in the form of hydraulic or pneumatic cylinder, and the motion direction of the shaft 130 is set in a direction parallel with the rotation center axis of the motion part 120. In a case where the shaft 130 moves forward by driving of the second driver 150, the surgical instrument 180 disposed within the accommodating groove 121 of the first position P1 deviates from the accommodating groove 121, thus moving to a second position P2 located in front of the first position P1, and in a case where the shaft 130 moves backward by driving of the second driver 150, the surgical instrument 180 disposed in the second position P2 moves to the first position P1, and is inserted into the accommodating groove 121 of the motion part 120. Meanwhile, in the process of moving forward and backward of the shaft 130, it is desirable that the second driver 150 stops for a moment in the first position P1 and then move again for the sake of smooth coupling or uncoupling of the front end of the shaft 130 and the rear end of the surgical instrument 180.

The third driver 160 is for providing driving force to the surgical instrument 180 that is coupled to the front end of the shaft 130 and moved to the second position P2. The third driver 160 may include the servo motor 161, and a driving wheel 162 that is disposed at a front end of the shaft 130 and that rotates by the driving force being provided from the servo motor 161. The third driver 160 may be provided in plural number for various operations. Further, although not illustrated in the drawings, on a coupling surface of the shaft 130 and the surgical instrument 180, a terminal for mutual electrical connection for transmitting power source or signals may be further provided.

The inputter 170 is provided on the base 110 such that the operator can input controls signals for operation or replacement. The inputter 170 includes a first inputter 171 that may generate control signals for driving the third driver 160 and a second inputter 172 that may generate control signals for driving the first driver 140 and the second driver 150.

The first inputter 171 may include one or more of various input devices such as a joystick, glove, trigger-gun, and manual controller that may generate control signals for driving the third driver 160 according to the manipulation of the operator.

The second inputter 172 may include a voice inputter 170 for recognizing the voice of the operator, and a voice recognizer that may generate control signals for driving the first driver 150 and the second driver 150 according to the voice input through the voice inputter 170. Meanwhile, although in the present embodiment, an example was explained where the first driver 140 and the second driver 150 are controlled by recognizing the voice of the operator, there is no limitation thereto, and thus it will also be possible to generate the control signals through a button that may be directly manipulated by the operator such as the first inputter or a sensor that may sense the operations of the operator.

The surgical instrument 180 may include a body part, an end effector that is provided at a front end of the body part, a following wheel 181 provided at a rear end of the body part, and a driving force transmission mechanism provided inside the body part to connect the following wheel 181 and the end effector, and the body part may have a joint. Such a surgical instrument 180 may be divided into forceps, electric cautery instruments, suture instruments, ultrasonic cutter instruments, washing and drainage instruments, etc., depending on the shape of the end effector, and may each be accommodated in the accommodating groove 121 of the motion part 120 by different type. The following wheel 181 is provided on the rear end of the surgical instrument 180 in a position corresponding to the driving wheel 162 of the third driver 160, and is configured to interlock with the driving wheel 162 to receive the driving force being provided from the third driver 160.

The multi-arm support arm 190 is connected to the base 110 at one end, and the other end is connected to the structure such as a wall, ceiling, and bottom, to support the base, and may include a gravity compensation mechanism for compensating the gravity by self-weight of the robot arm.

Hereinbelow, operations of the first embodiment of the wearable surgical robot arm will be explained.

The base 110 may be fixed to the arm of the operator and move together with the arm. The base 110 is supported against the structure such as the ceiling, wall and bottom inside the surgery room by the multi-joint support arm 190. Therefore, in the surgery room, with the operator wearing the surgical robot arm, the operator may manipulate the first inputter 171 provided in the base 110 to control the surgical instrument 180 mounted onto the robot arm, thereby proceeding with the surgery, and input a voice to the second inputter 172 to easily replace the surgical instrument 180 mounted onto the robot arm. Especially, since the operator may take off the robot arm and treat the surgical site directly with hands, when a dangerous situation occurs, immediate response is also possible.

The motion part 120 is rotatably installed in the base 110 so that it may be rotated by the first driver 140, and in the plurality of accommodating grooves 121 formed in the motion part 120, different types of surgical instruments 180 are inserted, respectively. When the operator makes a verbal instruction using voice to use a surgical instrument 180 that the operator wants to use during the surgery, the second driver 150 is driven backward to move the shaft 130 backward, thereby positioning the surgical instrument 180 of the second position P2 that is in use to the accommodating groove 121 of the first position P1, and the first driver 140 is driven to move the accommodating groove 121 to which the surgical instrument 180 is inserted corresponding to the corresponding voice instruction made, to the first position P1, and then the first driver 140 is driven forward to drive the shaft 130 forward, thereby moving the surgical instrument 180 disposed in the first position P1 to the second position P2.

As aforementioned, according to the present embodiment, replacement of the surgical instrument 180 may be made with only the voice of the operator, and thus it is not only possible to reduce the surgery time, but also additional workforce for replacing the surgical instrument 180 is not required. Further, since the surgeon may pre-configure a combination of the instruments necessary for the scheduled surgery, and dispose the accommodating groove 121 of the motion part 120 according to the order of use, the time spent on replacing the surgical instrument 180 may be minimized.

As aforementioned, in the surgical instrument 180 disposed in the second position P2, operation of the end effector is controlled as the operator manipulates the first inputter 171, and thus controlling the driving of the third driver 160. Therefore, a surgery may be proceeded using the end effector of the surgical instrument 180.

Especially, in a case of providing the first inputter 171 in a similar form as a manipulating part of a laparoscopic surgical instrument, a surgery may be proceeded in a form that is similar to a laparoscopic surgery, with the robot arm of the present embodiment mounted onto the arm of the operator, and thus, an operator experienced in laparoscopic surgery may use the robot arm of the present embodiment intuitively.

The scope of the present invention is not limited to the above-described embodiments, but may be implemented in various forms within the scope of the attached claims. Without departing from the gist of the present disclosure claimed in the claims, any person of ordinary skill in the art to which the present invention pertains is considered to be within the scope of the description of the claims of the present disclosure to various ranges that can be modified.

What is claimed is:

1. A wearable surgical robot arm comprising:
a base configured to move together with an arm of an operator;
a motion part that has a plurality of accommodating grooves in which a surgical instrument is be mounted and that is moveably installed on the base such that one of the plurality of accommodating grooves is positioned in a first position;
a shaft that is disposed such that the shaft moves forward or backward to selectively penetrate the accommodating groove positioned in the first position at a rear of the motion part;
a first driver that moves the motion part such that one surgical instrument selected from the surgical instruments mounted in the plurality of accommodating grooves moves to the first position;
a second driver that moves the shaft to move the surgical instrument positioned in the first position to a second position positioned in front of the first position; and
a third driver that provides driving force to the surgical instrument that moved to the second position.

2. The wearable surgical robot arm according to claim 1,
wherein the motion part is rotatably disposed on the base.

3. The wearable surgical robot arm according to claim 1,
wherein the third driver is provided in the shaft.

4. The wearable surgical robot arm according to claim 1,
wherein at a front end of the shaft, a driving wheel is provided for transmitting the driving force being provided from the third driver, and at a rear end of the surgical instrument, a following wheel is provided in a position correspond to the driving wheel to interlock with the driving wheel.

5. The wearable surgical robot arm according to claim 1,
wherein the base is provided with an inputter by which the operator inputs a control signal.

6. The wearable surgical robot arm according to claim 5,
wherein the inputter includes a first inputter for inputting a control signal for controlling operations of the surgical instrument.

7. The wearable surgical robot arm according to claim 5,
wherein the inputter includes a second inputter for inputting a control signal for driving the first driver and the second driver.

8. The wearable surgical robot arm according to claim 7,
wherein the second inputter includes a voice inputter for recognizing voice of the operator, and a voice recognizer for generating a control signal for driving the first driver and the second driver according to the voice input through the voice inputter.

9. The wearable surgical robot arm according to claim 1,
further including a multi-joint support arm that supports the base in a moveable form.
